(19) United States
(12) Patent Application Publication
(10) Pub. No.: US 2020/0150772 A1
(43) Pub. Date: May 14, 2020

(54) Sensing Hand Gestures Using Optical Sensors

(71) Applicant: Google LLC, Mountain View, CA (US)

(72) Inventors: Philip Quinn, San Jose, CA (US); Zhuangdi Zhu, East Lansing, MI (US)

(21) Appl. No.: 16/243,767

(22) Filed: Jan. 9, 2019

Related U.S. Application Data
(60) Provisional application No. 62/757,973, filed on Nov. 9, 2018.

(51) Int. Cl. G06F 3/01; G01P 15/18; A61B 5/1455; A61B 5/00

(52) U.S. Cl. CPC G06F 3/017; G01P 15/18; A61B 2562/0219; A61B 5/7203; A61B 5/1455

(57) ABSTRACT

The present disclosure provides for one-handed, touch-free interaction with smartwatches. An optical sensor in the smartwatch detects a user's hand and finger movements on the arm wearing the smartwatch. The gesture detection and recognition approaches in this design are lightweight and efficient to operate on small devices.

### CROSS-REFERENCE TO RELATED APPLICATIONS

The present application claims the benefit of the filing date of U.S. Provisional Patent Application No. 62/757,973 filed Nov. 9, 2018, the disclosure of which is hereby incorporated herein by reference.

### BACKGROUND

Interacting with a smartwatch typically requires the use of both hands/arms. For example, while the watch is worn on one arm, the hand on the other arm interacts with the screen or buttons on the watch. This bi-manual requirement often makes interaction awkward and difficult, thereby reducing the convenience of the smartwatch. For example, if the opposing hand is holding an object or is inaccessible, such as while holding coffee, holding an umbrella, pushing a stroller, etc, interaction is constrained to be passive glancing or reading. Similarly, due to the small size and fidelity of the screen on a smartwatch, touch interactions are often cumbersome, involving multiple steps, or are error prone.

Smartwatches typically implement a limited set of one-handed interactions powered by an inertial measurement unit (IMU), such as an accelerometer, gyroscope, etc. The most common is a “lift to wake” gesture that activates the screen when the watch is lifted in a manner indicative of a user wanting to glance at the screen. Conversely, when the user drops their arm, the screen goes to sleep. Some smartwatches also implement navigation gestures, such as scrolling, that are performed by a user twisting their wrist. However, an IMU provides for sensing gestures that involve large physical movements of the watch/arm. This is undesirable in many use cases. For example, the gestures are not discreet and may be socially awkward, and they disrupt the user's focus on the watch.

Other types of sensors may not be practical for integration into smartwatches to detect gestures by the user. For example, cameras, electrocardiogram sensors, ultrasonic sensors, strain gauges, etc. are large and expensive to integrate. Moreover, such sensors generally require per-user and/or per-session calibration.

### BRIEF SUMMARY

There are many common use cases where a low-fidelity, discreet, single-handed gesture is desirable to interact with a smartwatch. For example, if a user receives a notification and wants to quickly dismiss it, they could perform a quick brushing gesture using fingers of the arm on which the watch is worn. Similarly, if they wanted to send a short reply to a message notification, they could clench their fist, dictate their response, and then release their fist to send it. The gestures may also be continuous, allowing a user to adjust a parameter. For example, the user may bend their fingers to adjust the volume of music playing on their phone, where the amount of finger movement determines the change in volume level. The present disclosure provides for integration of optical sensors into a smartwatch to achieve discreet, single hand control gestures. The optical sensors are small enough to integrate into the watch chassis, efficient to operate, tolerant to the position and fit of the watch, and usable without per-user or per-session calibration for a smooth user experience.

One aspect of the disclosure provides a method for detecting hand motions by a wearable device, including emitting, by a light source, light towards a user's skin, receiving, by an optical sensor, reflected light, reading, by one or more processors, raw sensor data from the optical sensor, filtering, by the one or more processors, the raw sensor data to reduce noise, identifying, by the one or more processors, features in the filtered signal that correspond to movements of the wrist, hand, or fingers. The method further includes matching, by the one or more processors, the identified features to a specific gestural action, and performing, by the one or more processors, an interface operation corresponding to the specific gestural action. The wearable device may be a smartwatch worn on the user's arm, wherein the light is reflected off target objects, such as hemoglobin, and non-target objects in the user's arm. The filtering may include high-pass filtering and low-pass filtering, and may in some instances further include a median filter adapted to reduce spikes in the raw sensor data. According to some examples, the method may further include receiving sensor data from an inertial measurement unit (IMU), wherein the filtering comprises using the sensor data from the IMU to filter out noise from the raw sensor data from the optical sensor. Identifying features may include changing points in the filtered sensor data. The matching may include matching the identified features to recorded features for particular gestures. The interface operation may control a function of the wearable electronic device.

Another aspect of the disclosure provides a wearable electronic device adapted to receive input from hand gesture of an arm on which the device is worn. The device includes a light source configured to light towards the user's arm, an optical sensor adapted to receive reflected light from the user's arm, a memory storing information regarding gestural actions and interface operations, and one or more processors in communication with the optical sensor and the memory. The one or more processors may be configured to receive raw sensor data from the optical sensor, filter the raw sensor data to reduce noise, identify features in the filtered signal that correspond to movements of the wrist, hand, or fingers of the arm wearing the device, match the identified features to a specific gestural action, and perform an interface operation corresponding to the specific gestural action.

Another aspect of the disclosure provides a system, including a photoplethysmogram (PPG) sensor adapted to be positioned in close proximity to a user's arm, an inertial measurement unit (IMU) adapted to be positioned in close proximity to the user's arm, wherein each of the PPG sensor and the IMU are configured to receive data signals measuring movements of the user's arm, and one or more processing units, configured to process the received data signals, such processing including extracting features indicating particular hand gestures, and correlate the detected features with input operations for the one or more processors.

### BRIEF DESCRIPTION OF THE DRAWINGS

FIG. 1 illustrates an example hand gesture according to aspects of the disclosure.

FIG. 2 illustrates another example hand gesture according to aspects of the disclosure.

FIG. 3 illustrates another example hand gesture according to aspects of the disclosure.

FIG. 4 illustrates another example hand gesture according to aspects of the disclosure.

FIGS. 5A-D illustrate various example positions of a wearable electronic device on a user's arm.

FIG. 6 is a block diagram illustrating an example system according to aspects of the disclosure.

FIG. 7 is a circuit diagram illustrating an example optical sensor according to aspects of the disclosure.

FIGS. 8A-B are example graphs illustrating received optical sensor signals for different hand status according to aspects of the disclosure.

FIG. 9 is an example graph illustrating received IMU signals with a steady arm according to aspects of the disclosure.

FIG. 10 is an example graph illustrating received IMU signals with a waving arm according to aspects of the disclosure.

FIGS. 11A-C illustrate example graphs for pre-processing a received optical sensor signal according to aspects of the disclosure.

FIG. 12 illustrates example graphs for pre-processing a received optical sensor signal when a wearable device is in a motionless state according to aspects of the disclosure.

FIG. 13 illustrates example graphs for pre-processing a received optical sensor signal when a user wearing the wearable device executes a gesture according to aspects of the disclosure.

FIG. 14 illustrates feature extraction graphs for a received optical sensor signal when the user wearing the device executes a gesture according to aspects of the disclosure.

FIG. 15A illustrates a received signal of hand gesture movements according to aspects of the disclosure.

FIG. 15B illustrates a received signal of random hand movements according to aspects of the disclosure.

FIGS. 16A-D illustrate example of changing points on signals of four different gestures according to aspects of the disclosure.

FIG. 17 is a flow diagram illustrating an example method according to aspects of the disclosure.

### DETAILED DESCRIPTION

The present disclosure provides for an optical sensor embedded into a watch chassis of a smartwatch, with an algorithm to sense gestures from movements of the user's fingers, wrist, and/or arm on the arm that is wearing the watch. The optical sensor is designed to be small and power-efficient, and the algorithm is designed to be robust to noise and tolerant to user variance. The optical sensor may include, for example, an LED and photodiode. For example, a photoplethysmogram (PPG) signal is used to detect specific physical movements/articulations of the fingers/arm/wrist, which are analyzed as gestures that invoke an action on the watch.

Raw sensor data from the photodiode is read and processed to reduce noise. Features in the processed data are identified, wherein such features correspond to movements of the wrist, hand, or fingers. A gesture detection algorithm matches these features, and potentially other signals, to a specific gestural action. An interface action is performed on the device that corresponds to the gestural action.

The raw sensor data may be read through an analogue-to-digital converter (ADC) to quantize the signal so that it can be digitally processed. This signal is then filtered for noise as a preprocessing stage. In some examples, some of this filtering may be performed on the analogue signal before the ADC.

The signal preprocessing may include a low-pass filter to remove high-frequency electrical noise, and a high-pass filter to remove the DC offset and drift. Sources of noise, offset, and drift may include leakage of light from external sources, such as if the watch is not worn tightly, reflectivity of the user's skin, which may vary by skin tone, and temperature of the sensor. The user's heart rate may also be a source of noise that is filtered out or suppressed, for example, by setting a cut-off frequency of the filters appropriately, or by using the calculated heart rate to set an adaptive band-stop filter. In one example, signal preprocessing includes applying a median filter to remove spikes from the signal, using a high-pass filter to filter out the baseline DC offset and drift from the signal, and using a low-pass filter to remove high-frequency noise and to further smooth the signal. The filters are configured such that hand gestures are not filtered out. Hand gestures are low-frequency movements, and an aggressive cut-off frequency may lead to a loss of features caused by hand gestures.

Features are extracted from the resultant preprocessed signal for changes or patterns indicative of wrist, hand, or finger movements. These features are extracted against a background of movements in other parts of the arm that are not of interest, such as bending of the elbow. Any of one or more various approaches may be used to identify these features, such as peak detection, signal variance, Haar-like feature encoding, frequency analysis (spectrogram), etc. Features may also be extracted from a first-order derivative of the signal, indicating a velocity of the motion, and a second-order derivative of the signal, indicating an acceleration of the motion. For each of the processed signal, the velocity of the signal, and the acceleration of the signal, features can be extracted from the time-domain and the frequency-domain. In general, amplitude changes, such as local maxima and minima, may be used to extract the time-domain features, and a short-time Fourier transform (STFT) may be used to extract the frequency-domain features.

In some cases, thresholds may be applied to these features to screen them for signals that are indicative of actual gestures. For example, local minima and maxima may only be considered if the difference with neighboring minima/maxima is greater than some threshold, thus filtering out points created by unintended movement and noise, and maintaining those representing significant shape changes caused by intended gestures. Further, features may be dependent on other feature thresholds. For example, local minima/maxima may only be considered if there is a spike in the variance of the signal. A spike in variance is indicative of an intentional user gesture, and therefore may be selected to create a window around which the algorithm will attempt to extract features and detect a gesture.

These features inform a gesture detection algorithm that identifies a complete, intentional gesture from a user. The detection algorithm may involve heuristic components, such as thresholds on the feature values or pattern matching metrics, and machine learning components that have been trained on feature samples from both intentional gestures and accidental noise. For example, the features may be reduced using a Linear Discriminant Analysis (LDA) or Quadratic Discriminant Analysis (QDA) to find the boundaries between different gesture classes.

When attempting to detect multiple gesture classes, or those that are not easily discriminable with heuristic methods, machine learning models may be trained on the above features to provide a gesture recognizer. Examples of such machine learning models may include K-Nearest Neighbor, Random Forests, Recurrent Neural Networks, etc. The detector may also use signals from other sensors to filter possible noise. For example, certain gestures may only be available when the user has already lifted their arm, which may be detected by an IMU, and the watch's screen is on.

The system described herein provides for improved user experience, as a user can discreetly manipulate a smartwatch using only the hand on which the smartwatch is being worn. For example, using finger or hand gestures, such as clenching a fist, moving a finger up and down, tapping fingers and thumb together, etc., the user may perform a variety of operations on the smartwatch.

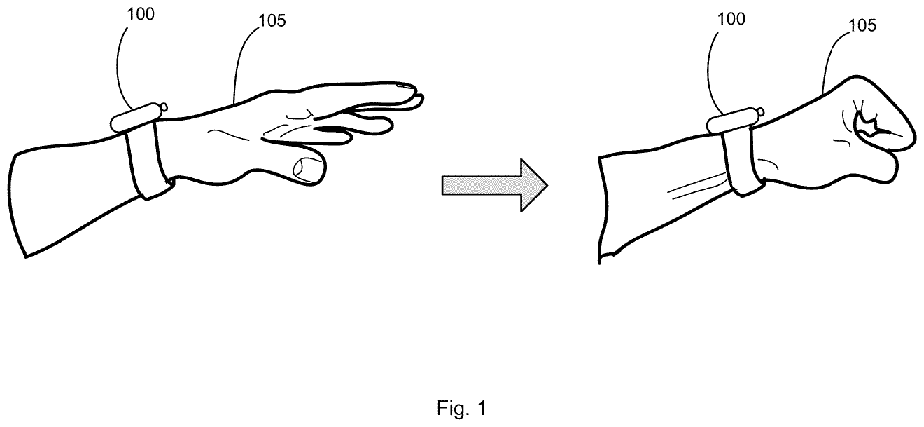

FIGS. 1-4 illustrates different example gestures. Each of the gestures may be performed with one hand 105 wearing a smartwatch 100 or other wearable computing device. Moreover, each of the gestures are relatively discreet, and are easy for users to accept and remember. While a few example gestures are shown, it should be understood that any number of additional one-handed gestures may also be recognized to perform a particular action on the smartwatch.

FIG. 1 illustrates a “hold” gesture, where the user clenches a first and keeps the fist closed. This may be used to, for example, activate a feature of the smartwatch, such as an assistant, a dictation application, a display of particular information, etc.

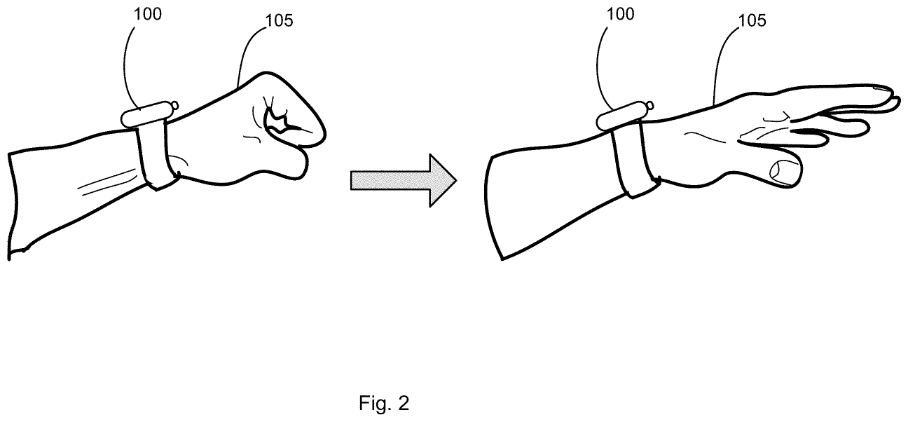

FIG. 2 illustrates a “release” gesture, where the user releases the fist. This may be used to, for example, dismiss a feature activated by the “hold” of FIG. 1.

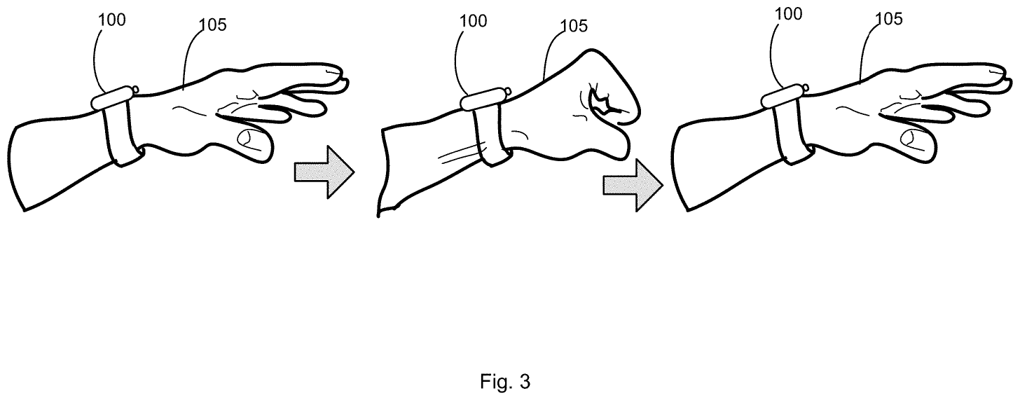

FIG. 3 illustrates a “squeeze” gesture, where the user clenches and quickly releases the fist. Just some examples of functions activated by the “squeeze” gesture may include activation or deactivation of triggers, such as opening or dismissing a notification, adjusting parameters, such as brightness of the display or volume of audio played through earbuds connected by Bluetooth, or any of a number of other features. According to some examples, a tightness of the squeeze may correspond to different functions. For example, a tight squeeze may trigger a more dramatic volume increase than a loose squeeze, etc.

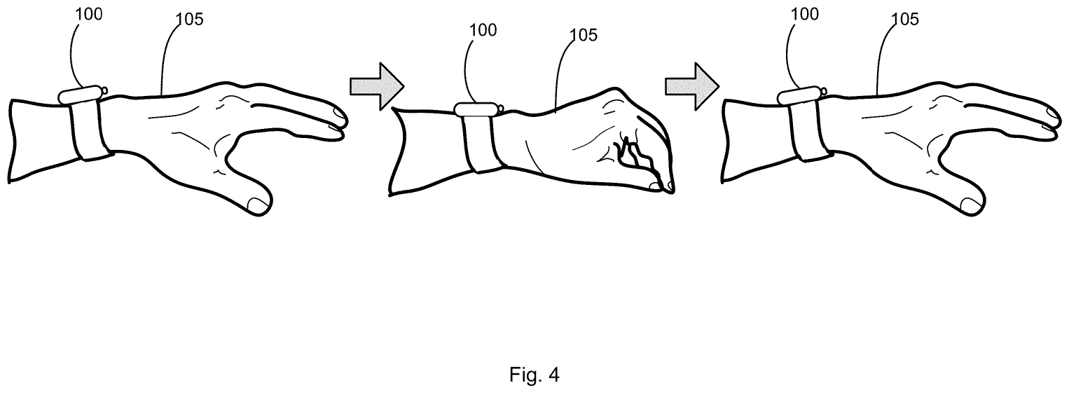

FIG. 4 illustrates a “tap” gesture, where the user taps one or more fingers with the thumb. The tap may be performed one or more times, for example, with different numbers of taps corresponding to different functions. Moreover, the user may tap different combinations of fingers, such as only the middle finger and thumb, all four fingers and the thumb, index and middle fingers and thumb, etc. Each different combination may correspond to a different function. Just some examples of functions activated by the “tap” gesture may also include activation or deactivation of triggers, or any of a number of other features.

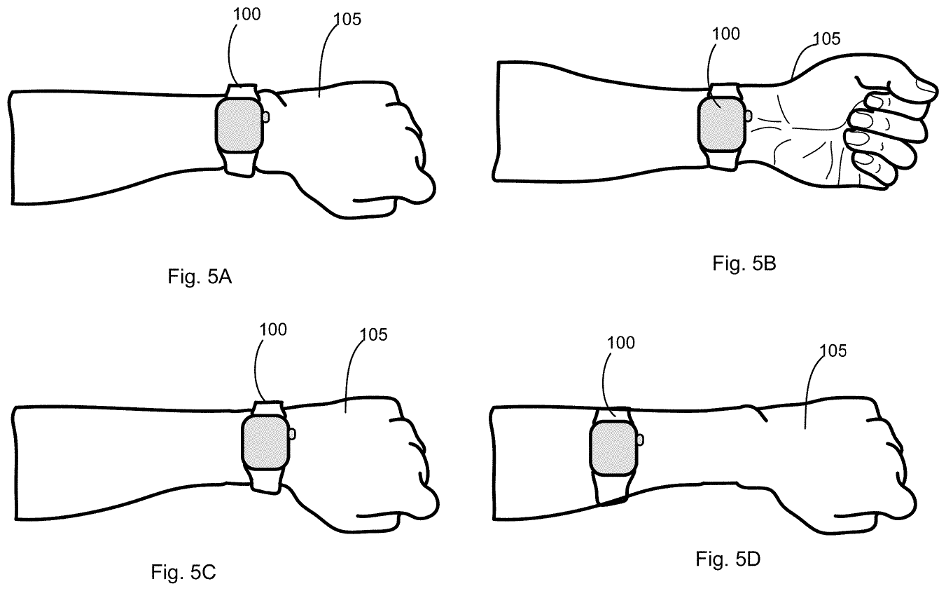

FIGS. 5A-D illustrate examples of variation in watch placement on the user's arm. In each illustration, a position of the watch varies, whether it is on an outside or inside portion of the user's arm, higher towards the user's shoulder, lower towards the user's hand, etc. The watch 100 may detect gestures regardless of how the watch is worn on the user's arm. However, in some instances, the signals may be different. The watch 100 may adapt in some cases to the different signals based on how the watch is worn. For example, the watch may determine a position on the user's arm, such as by manual user input, detection of light, heat, or other parameters, detection of a position of the watchband, such as which hole a clasp is secured to, or by any of a variety of other techniques. Further, the detection of gestures may be adjusted based on the determined placement on the user's arm. For example, if the watch 100 is worn in the position of FIG. 5C closer to a user's hand or palm, where there are more bones as compared to a standard outside arm position such as in FIG. 5A, processing of signals from the sensors may be performed according to different parameters such that gestures are not mistaken for noise.

According to other examples, the watch 100 may be worn tighter on one person's wrist than on another person's wrist. Wearing the watch looser may result in ambient light leakage. To account for such leakage, feature extraction may be modified to use features that are robust to light leakage. For example, the watch may detect a level of looseness or tightness on the user's wrist, and adapt the feature extraction techniques accordingly. In other examples, the watch may be shaped to have a convex bottom, so that space between user skin and the watch bottom is minimized regardless of the tightness of the watch band, thereby reducing ambient light leakage.

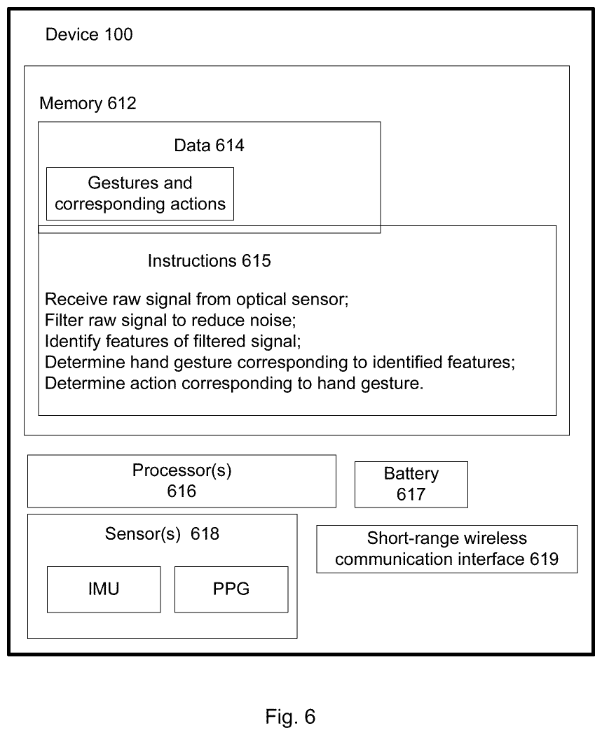

FIG. 6 illustrates an example of internal components of a wearable computing device, such as a smartwatch 100. While a number of internal components are shown, it should be understood that additional or fewer components may be included. By way of example only, the devices may include components typically found in wearable devices, such as speakers, microphones, displays, etc.

The smartwatch 100 may include one or more processors 616, one or more memory units 612, as well as other components. For example, the device 100 may include one or more sensors 618, wireless pairing interface 619, and a battery 617.

The memory 612 may store information accessible by the one or more processors 616, including data 614 instructions 615 that may be executed or otherwise used by the one or more processors 616. For example, memory 612 may be of any type capable of storing information accessible by the processor(s), including a computing device-readable medium, or other medium that stores data that may be read with the aid of an electronic device, such as a volatile memory, non-volatile as well as other write-capable and read-only memories. By way of example only, memory 612 may be a static random-access memory (SRAM) configured to provide fast lookups. Systems and methods may include different combinations of the foregoing, whereby different portions of the instructions and data are stored on different types of media.

The data 614 may be retrieved, stored or modified by the one or more processors 616 in accordance with the instructions 615. For instance, data 614 may include a correlation of detected features with particular gestures, a correlation of gestures with actions to be taken by the smartwatch 100, and/or any of a variety of other types of data. Although the claimed subject matter is not limited by any particular data structure, the data may be stored in computing device registers, in a relational database as a table having a plurality of different fields and records, XML documents or flat files. The data may also be formatted in any computing device-readable format.

The instructions 615 may be any set of instructions to be executed directly (such as machine code) or indirectly (such as scripts) by the one or more processors 616. For example, the instructions may be stored as computing device code on the computing device-readable medium. In that regard, the terms “instructions” and “programs” may be used interchangeably herein. The instructions may be stored in object code format for direct processing by the processor, or in any other computing device language including scripts or collections of independent source code modules that are interpreted on demand or compiled in advance. The instructions 615 may be executed to detect a gesture using signals from the sensors 618, determine an action corresponding to the detected gesture, and perform the action. Functions, methods and routines of the instructions are explained in more detail below.

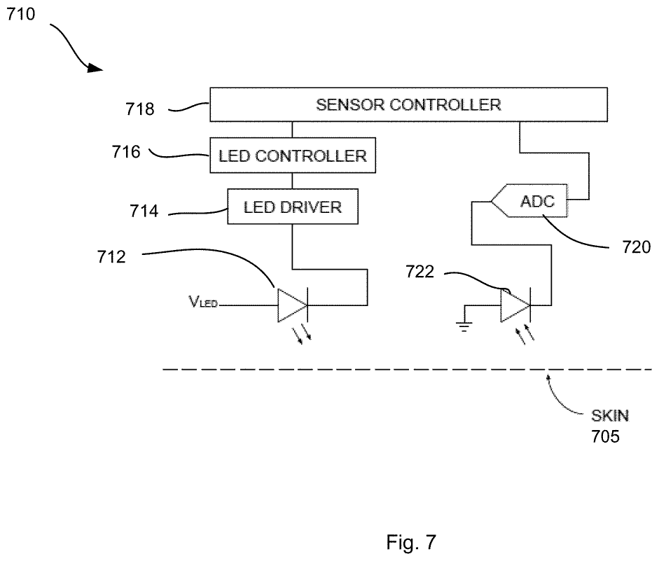

The one or more processors 616 may be microprocessors, logic circuitry (e.g., logic gates, flip-flops, etc.) hard-wired into the device 100 itself, or may be a dedicated application specific integrated circuit (ASIC). It should be understood that the one or more processors 616 are not limited to hard-wired logic circuitry, but may also include any commercially available processing unit, or any hardware-based processors, such as a field programmable gate array (FPGA). In some examples, the one or more processors 616 may include a state machine. The processors 616 may be configured to execute the instruction 615 to, for example, perform a method such as described below in connection with FIG. 7.

The one or more sensors 618 may include any of a variety of mechanical or electromechanical sensors for detecting gestures. Such sensors may include, for example, an IMU, an optical sensor, such as a photoplethysmogram (PPG), etc. According to some examples, the sensors 618 may further include an accelerometer, gyroscope, barometer, audio sensor, vibration sensor, heat sensor, radio frequency (RF) sensor, etc.

The short range wireless pairing interface 619 may be used to form connections with other devices, such as a smartphone, earbuds, etc. The connection may be, for example, a Bluetooth connection or any other type of wireless pairing. By way of example only, connections with other devices may include an ACL link.

Although FIG. 6 functionally illustrates the processor, memory, and other elements of device 100 as being within the same block, it will be understood by those of ordinary skill in the art that the processor and memory may actually include multiple processors and memory units that may or may not be stored within the same physical housing. For example, memory 612 may be a volatile memory or other type of memory located in a casing different from that of computing device 100. Moreover, the various components described above may be part of one or more electronic devices. By way of example only, the smartwatch 100 may detect signals and communicate information regarding the detected signals to a remote server, and receive corresponding information, such as which corresponding action to perform.

FIG. 7 illustrates an example PPG sensor 710, which may be used to detect discreet, single-hand gestures. The PPG 710 includes a light source 712 which illuminates a user's skin 705, and a photodiode 722 which measures changes in light reflection and absorption. When a user moves his fingers, it twists the veins that flow through the arm, and produces substantial changes to pressure and blood volume in the arm. This is reflected in a direct current (DC) component of the PPG signal. The changes in pressure and blood volume measured by the PPG sensor 710 may be analyzed to determine a corresponding gesture and action to be taken. For example the measurements received by the photodiode 722 may be converted into a digital signal by analog-to-digital converter 720, and processed by sensor controller 718 or a processing unit (not shown) in communication with the sensor controller 718.

The light source 712 may be, for example, a light emitting diode (LED), activated using an LED driver 714 and an LED controller 716. The LED controller 716 may further be controlled by sensor controller 718. The light emitted from the light source 712 should be strong enough to penetrate into the skin 705. A wavelength of the light may be chosen to maximize the amount of light reflected from a target object and minimize the amount of light reflected by non-target objects. For measuring a heart rate, for example, the target object is hemoglobin in the user's arteries, and the non-target objects are ambience and surrounding skin, muscle, and bone. According to some examples, a narrow spectrum of green (˜550 nm), red (˜660 nm), or infrared (˜940 nm) light may be chosen for this purpose.

In the case of detecting the user's heart rate, each cardiac cycle of the heart produces a pulse of pressure to move blood from the heart through the arteries. When observing a single point in an artery, this pulse produces a spike in blood volume as it is pushed through. This change in blood volume produces a commensurate change in the amount of light it reflects from the LED 712 to the photodiode 722. The frequency of these changes, which make up the AC component of the signal, can be analyzed as the user's heart rate. However, as arteries are continuous vessels without valves or switches, any constriction or relaxation to an artery produces a change in pressure characteristics throughout its entire path. For example, if an artery is pinched, the pressure on the side coming from the heart will increase as blood volume builds. Conversely, the pressure will fall on the other side. Changes in the arteries that flow through the arm, into the hand, and to the fingers are analyzed to determine gestures made by a user's hand. The movement of the hand, fingers, and wrist twists and bends these arteries, producing substantial changes to the pressure and blood volume in the arm. These changes from movements in the hand appear as fluctuations in the DC component of the signal from the photodiode 722, and are aggressively filtered out by applications that seek to analyze the heart rate.

The raw sensor data may be read through the ADC 720 to quantize the signal so that it can be digitally processed. This signal is then filtered for noise as a preprocessing stage. In some examples, some of the filtering may be performed on the analogue signal before the ADC 720.

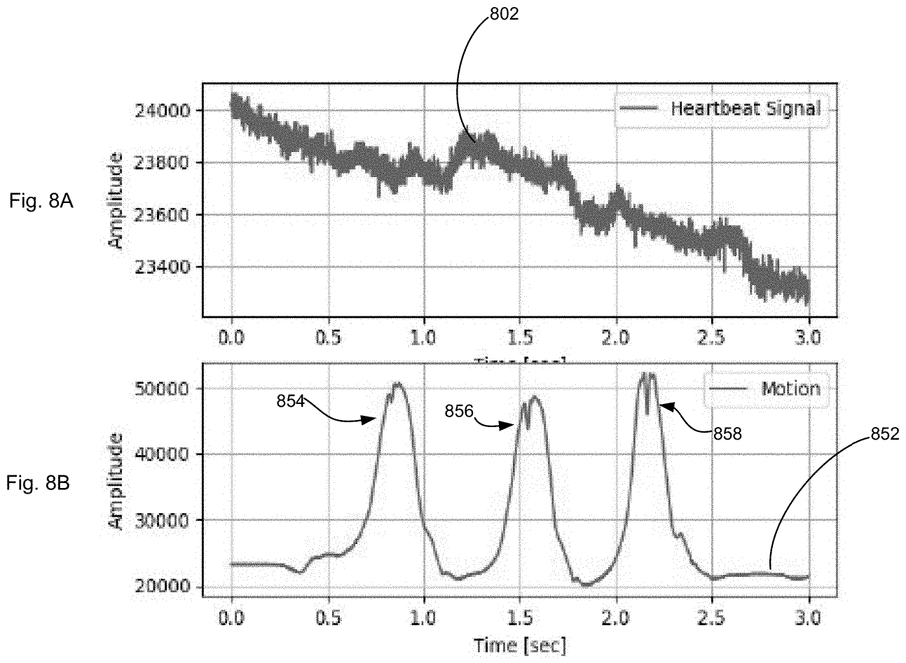

FIGS. 8A-B illustrates two examples of the received PPG signal. In FIG. 8A, the PPG signal 802 is collected when the user arm is steady. In FIG. 8B, the PPG signal 852 is collected when the user is clenching his fist three times. Each clench and release may be observed as a rise and fall of the signal 852. For example, a first rise 854 is observed around approximately 0.8 s, a second rise 856 is observed around approximately 1.5 s, and a third rise 858 is observed around approximately 2.2 s.

The IMU sensor, mentioned above with respect to FIG. 6, generates a three-dimensional signal which provides information about the direction and speed of the sensor movement. Features may be extracted from the IMU signal to help determine whether arm or wrist movement is involved when the signal is collected. When using discreet, all hand gestures such as those described above in FIGS. 1-4, rather than gestures involving movement of the arm or the wrist, significant IMU signal changes will infer that the user's arm or wrist is in an activity mode, and therefore the user is not intentionally performing hand gestures. For example, features from the IMU signal may be used to filter out noise motions from real gestures.

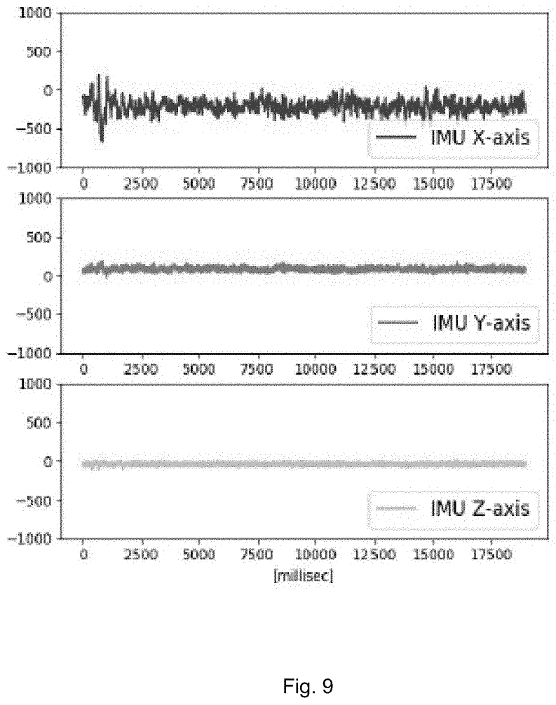

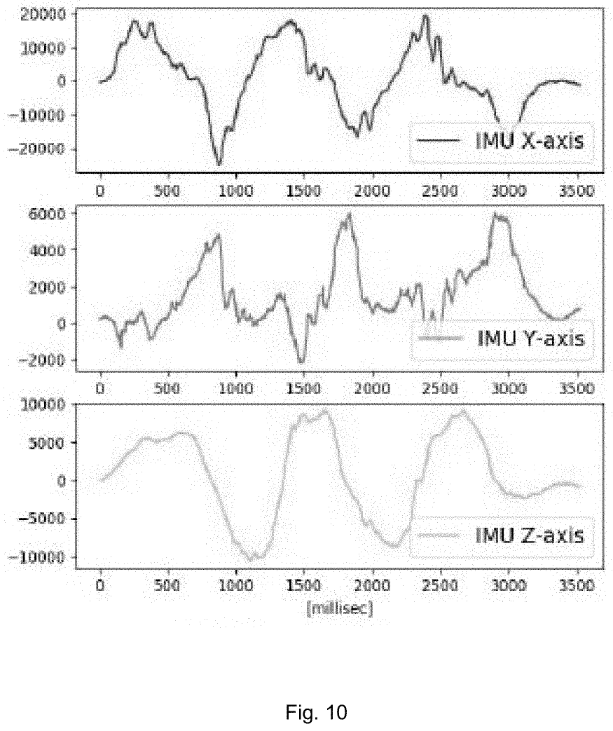

FIGS. 9-10 show two examples of the received IMU signal. In FIG. 9, the IMU signal is collected when the user's arm is steady. In each of the x, y, and z directions, the IMU is relatively steady. In FIG. 10, the IMU signal is collected when the user is waving randomly. In contrast to FIG. 9, the IMU signal in each of the x, y, and z directions in FIG. 10 fluctuates significantly.

PPG signals may be noisy due to various factors, such as electrical noise, light leakage, skin color, or the user's heartbeats. In some examples, the PPG signal may be pre-processed to reduce such noise. For example, a high-pass filter may be used to filter out baseline DC offset and drift from the signal. Alternatively or additionally, a low-pass filter may be used to remove high-frequency noise and to further smooth the signal.

A high-pass filter passes signals of frequency higher than a cutoff frequency. By way of example, a resistor-capacitor (RC) high-pass filter computes output samples yᵢby adding the difference between the last two input samples x₁ and xᵢ₋₁ to the last output sample yᵢ₋₁and weighting them by some damping parameter α. Given the sampling frequency fq of the signal, and a desired cutoff frequency c, an input signal x is filtered as follows:

yᵢ=α(yᵢ₋₁+xᵢ−xᵢ₋₁),

where α is the ratio of two components: α=1/(2π·c)]/[1/(2π·c)+1/fq]. Other implementations may use, for example, finite impulse response (FIR) or infinite impulse response (IIR) discrete-time filters to achieve a similar response.

A low pass filter only passes signals with a frequency that is lower than a cutoff. The low-pass filter may be implemented, for example, by doing a convolution between the input signal x and a coefficient window w which moves through the input signal:

$${y}_{i} = {\sum }_{k = 1}^{I} {w}_{k} {x}_{i - k} ,$$

where w contains the 1 weights of the filter coefficients selected for the desired cutoff frequency.

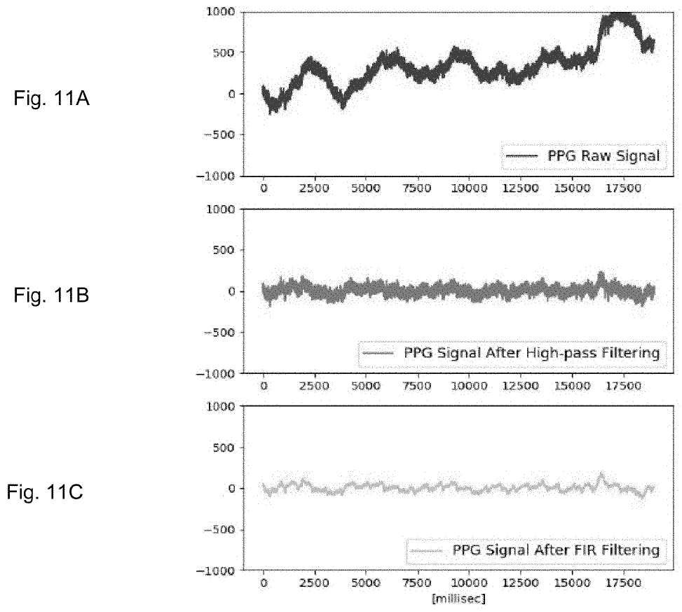

FIGS. 11A-C show examples of how a PPG signal is pre-processed using these filters. The raw PPG signal, shown in FIG. 11A, is received when the sensor is worn by a user in a motionless state. This raw PPG signal shows only the user's heartbeat signal. After high-pass filtering, shown in FIG. 11B, the drift and offset is removed so that the signal converges towards zero. After the low-pass filtering, shown in FIG. 11C, small jitters caused by high-frequency noise are filtered out.

According to some examples, signal pre-processing may include three steps. For example, a median filter may first be applied to remove spikes from the signal. The high-pass filter may be applied next to filter out the baseline DC offset and drift, followed by the low-pass filter to remove high-frequency noise and to further smooth the signal.

The filters should be configured such that hand gestures are not filtered out. Specifically, hand gestures are low-frequency movements, and an aggressive cut-off frequency may lead to a loss of features caused by hand gestures.

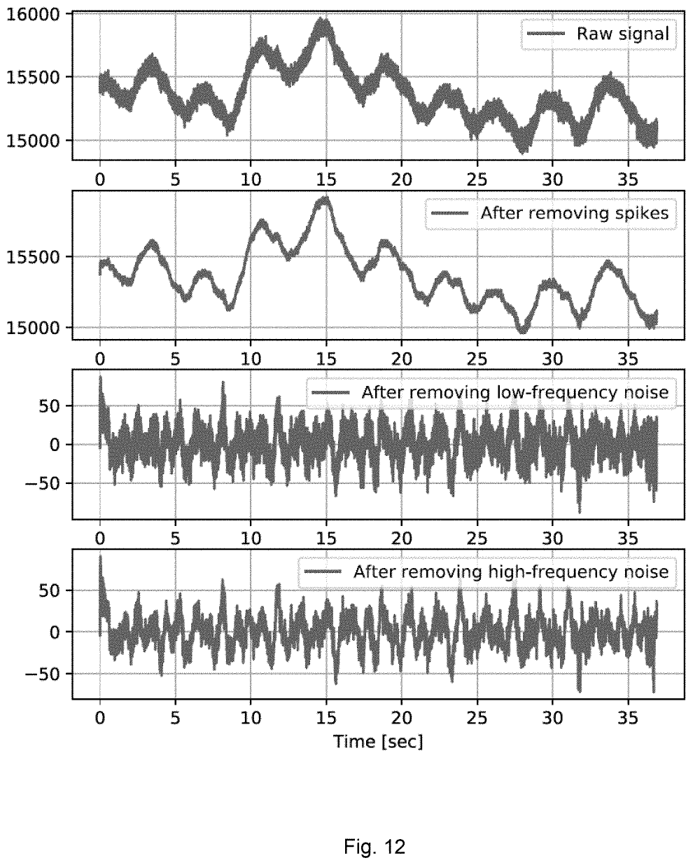

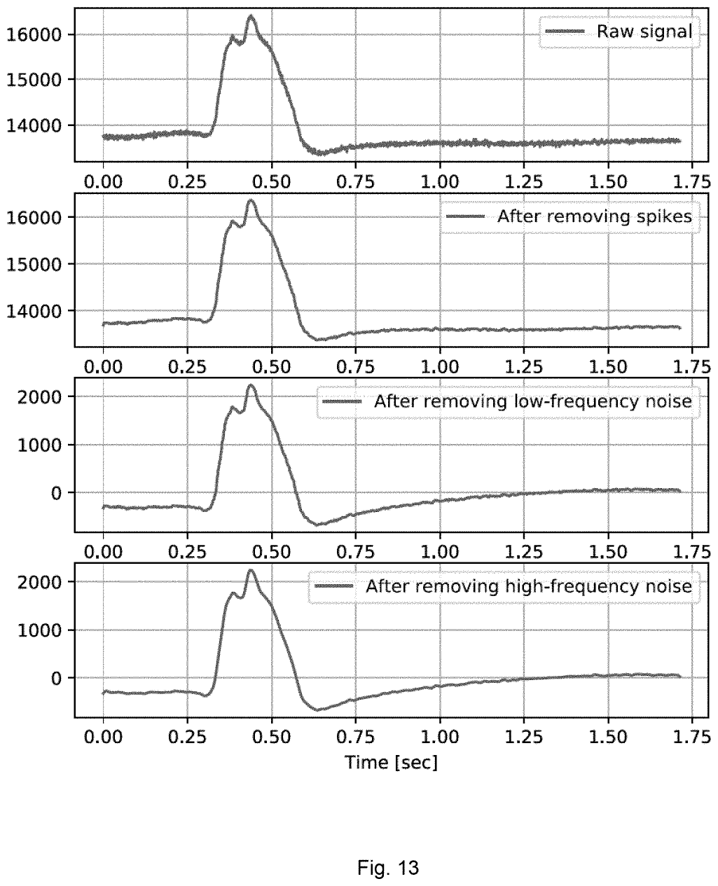

FIGS. 12 and 13 show examples of how a PPG signal is pre-processed using three-step filtering. FIG. 12 shows a sensor signal when it is worn by a user in a motionless state, showing only their heartbeat signal. FIG. 13 shows a sensor signal when the same user clenches their fist and quickly releases it. The heartbeat signal shown in the raw signal and after removing spikes in FIG. 12 is removed during the removal of low-frequency noise and high-frequency noise in FIG. 12. Moreover, as seen in FIG. 13 after low- and high-frequency noise filtering, the signal converges towards zero. The intentional first gesture of FIG. 13 is clearly visible against the background noise.

Features are extracted from the filtered signal for changes or patterns indicative of wrist, hand, or finger movements. The features are a reduction of the waveform shape that are informative of aspects of wrist, hand, or finger movements. For example, features may include the temporal pattern of local minima/maxima in a signal (wavelet analysis), the magnitude of a signal between local minima/maxima, the peak components of the signal's spectrogram (Fourier analysis). In some examples, rather than identifying specific movements, such as bending the index finger vs. bending the wrist, classes of movements may be identified, such as bending up, bending down, twisting, rubbing, etc. The features are extracted against a background of movements in other parts of the arm that are not of interest, such as bending the elbow. In some examples, multiple approaches may be used to identify these features. For example, any combination of peak detection, signal variance, Haar-like feature encoding, and frequency analysis/spectrogram may be used.

Features may also be extracted from a first-order derivative of the signal, indicating a velocity of the motion, and/or a second-order derivative of the signal, indicating an acceleration of the motion.

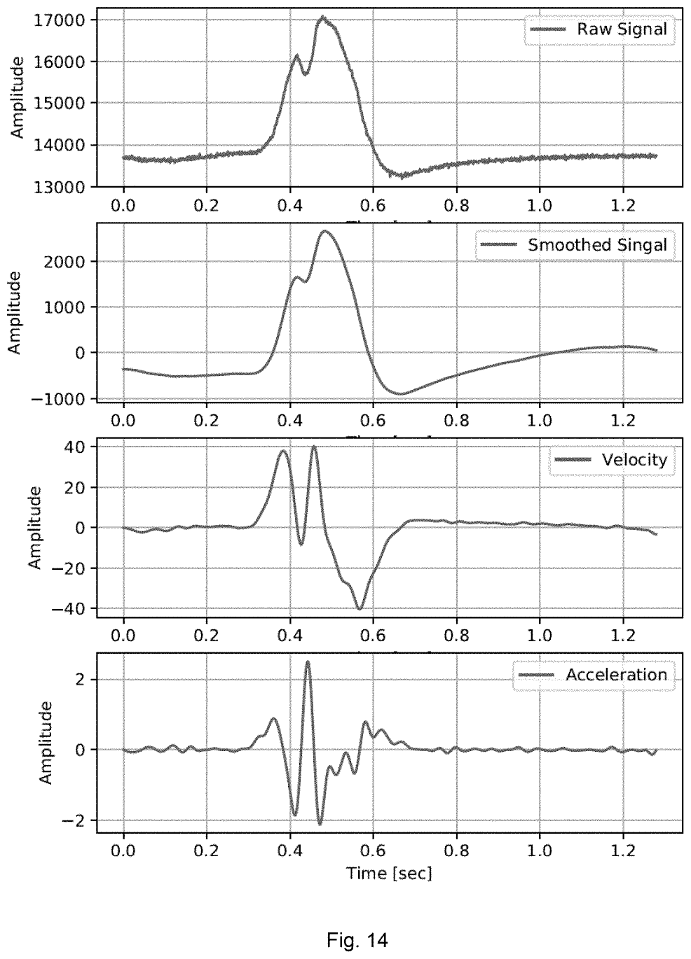

FIG. 14 shows an example of the raw, processed, velocity, and acceleration signals when a user squeezes and releases their fist. For each of the three sequences, including the processed signal, the velocity of the signal, and the acceleration of the signal, features can be extracted from the time-domain and the frequency-domain. Amplitude changes, such as local maxima and minima, may be used to extract the time-domain features. A short-time Fourier transform (STFT) may be used to extract the frequency-domain features.

In some examples, thresholds may be applied to these features to screen them for signals that are indicative of actual gestures. For example, local minima and maxima may be considered if the difference with neighboring minima/maxima is greater than some threshold. As such, points created by unintended movement and noise may be filtered out, while maintaining points representing significant shape changes caused by intended gestures.

Features may also be dependent on other feature thresholds. For example, local minima/maxima may be considered if there is a spike in the variance of the signal. A spike in variance is indicative of an intentional user gesture, and therefore may be selected to create a window around which the algorithm will attempt to extract features and detect a gesture. By way of example only, a window including signal data 500 ms before and 1500 ms after the spike may be analyzed to determine the gesture.

The extracted features inform a gesture detection component that identifies a complete, intentional gesture from a user. The gesture detection component detects the moment when a hand gesture occurs. In some examples, a user may be provided with early feedback once the user starts a gesture. For example, a motion detection model may respond immediately after it detects any abrupt changes in the received PPG/IMU signal, which may occur before a gesture completes. The motion detection model may further filter out noise caused by unintended hand motions, and only signal sequences caused by significant hand motions can pass. Therefore, the motion detection model reduces the burden of the gesture recognition component.

The detection component may involve heuristic components, such as thresholds on the feature values, and machine learning components that have been trained on feature samples from both intentional gestures and accidental noise. For example, the features described above may be reduced using a Linear Discriminant Analysis (LDA) or Quadratic Discriminant Analysis (QDA) to find the boundaries between different gesture classes. When attempting to detect multiple gesture classes, or those that are not easily discriminable with heuristic methods, machine learning models, such as K-Nearest Neighbor, Random Forests, and Recurrent Neural Networks, may be trained on the above features to provide a gesture recognizer.

According to some examples, gesture detection may include obtaining a velocity of a PPG signal, calculating a moving variance of the velocity, calculating the proportional change of the moving variance, and detection of the gesture.

A velocity sequence may be obtained by taking a first order derivative of the PPG signal. The velocity sequence may represent a change in status of the user's hand, such as a change from a motionless state to a moving state, or a change from slow movement to quick movement.

To calculate the moving variance, a sliding window may be used to traverse through the filtered signal, and the variance of the velocity may be calculated in each timing window. The window may be set to, for example, 200 ms with a stride of 50 ms. It should be understood that various window settings may be used.

The proportional change of the moving variance may be calculated based on the change in variance in each time window compared to historical status.

When a gesture occurs, it comes with a rapid change in the signal velocity, and the proportional changes will be higher than when there is no gesture. Accordingly, a threshold may be set for the proportional changes, such that a hand gesture is detected above the threshold.

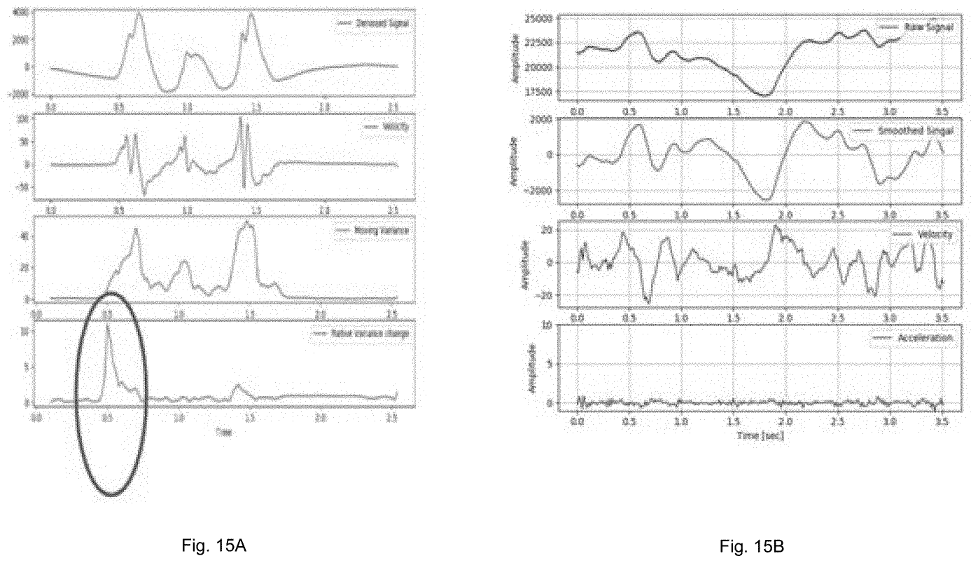

FIGS. 15A illustrates a received signal of hand gesture movements, while FIG. 15B illustrates a received signal of random hand movements. Each figure contains the raw signal, the velocity of the signal, the moving variance of the velocity, and the proportional change of the variance. In FIG. 15A, the start of a hand gesture, such as a clench and release, is identified at approximately 0.5 seconds. In FIG. 15B, although there appear to be random changes in the raw signal, no gestures are detected because the proportional change over time is lower than the threshold, which is set at 5.

Once a potential gesture is detected at time t, signal sequence that contains time t may be used to further recognize this gesture. Features are extracted from the PPG signal and the IMU signal, and used to recognize a gesture.

PPG signal sequences that have passed the gesture detection model may be used as inputs to feature generation algorithms. Once the gesture detection model has detected a hand motion at time t, a PPG signal sequence that contains this time t is extracted and used as raw inputs to the feature generation model. For example, a signal sequence x and velocity sequence v may be used to generate PPG features.

Various metrics may be calculated from the PPG signal sequence x and velocity sequence v. Examples of such metrics include root mean square of sequence x, average of sequence x, kurtosis of sequence x, skewness of sequence x, etc. These metrics are generally time-independent.

Time dependent features may be obtained using a peak detection feature extraction algorithm, on the PPG signal sequence x, for example. According to this algorithm, local maxima and minima are identified, noted as changing points representing abrupt changes in the sequence x. Each changing point is traversed to get the vertical distance with its neighboring changing point. The distances are sorted, and pairs of changing points forming the top distances are kept, while the others may be discarded. This process helps filter out small jitters in the signal sequence, so that only the significant shape changes will be reserved as features.

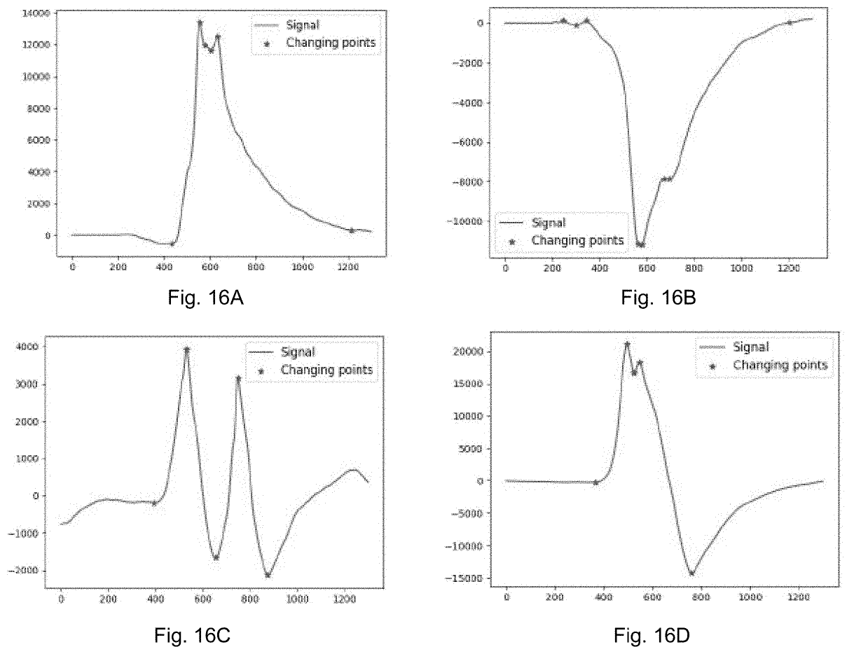

FIGS. 16A-D illustrate four examples of signals for gestures and the changing points related to the top four distances for each gesture. FIG. 16A illustrates an example signal for a “hold” gesture, such as illustrated in FIG. 1. FIG. 16B illustrates an example signal for a “release” gesture, such as illustrated in FIG. 2. FIG. 16C illustrates an example signal for a “tap” gesture, such as illustrated in FIG. 4. FIG. 16D illustrates an example signal for a “squeeze” gesture, such as illustrated in FIG. 3. A heuristic pattern recognition algorithm or machine learning model may be trained to recognize gestures based on a shape of the signal, such as based on relative positions of the changing points.

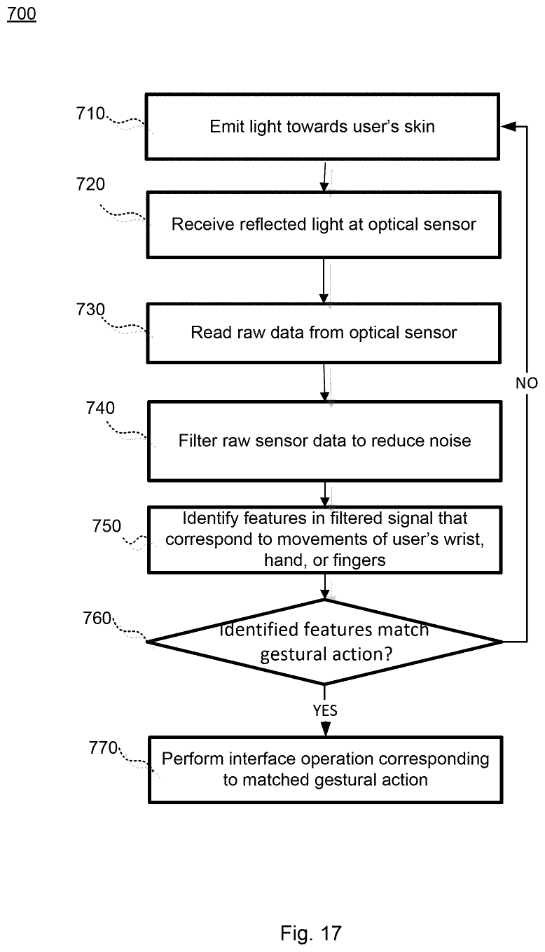

FIG. 17 is a flow diagram illustrating an example method 700 of detecting one-handed gestures by a wearable device. The wearable device may be a smartwatch or other wearable electronic device, such as a fitness tracker, gloves, ring, wristband, etc. with integrated electronics. While the operations are illustrated and described in a particular order, it should be understood that the order may be modified and that operations may be added or omitted.

In block 710, light is emitted towards the user's skin. For example, the light may be from a light source on the wearable device. According to some examples, the light source may be an LED in an optical sensor, such as a PPG sensor, in the wearable device. The light may penetrate the user's skin, for example, to reach the user's blood vessels, arteries, muscle tissue, etc.

In block 720, reflected light is received at the optical sensor. For example, the light may be reflected off the user's skin, blood vessels, hemoglobin, etc. The reflected light may have different intensity, frequency, or other characteristics based on what it reflected off. For example, it may have reflected off a target object, such as hemoglobin, or a non-target object, such as anything else.

In block 730, the raw sensor data is read from the optical sensor. For example, one or more processors may read the raw sensor data. The raw sensor data may take the form of a signal have a particular shape.

In block 740, the raw sensor data is filtered to reduce or eliminate noise. For example, a median filter may reduce spikes. A high-pass filter may filter out baseline DC offset and drift from the signal. A low-pass filter may remove high-frequency noise and further smooth the signal. One, all, or any combination of such filters may be used.

In block 750, features are identified in the filter signal, wherein the features correspond to movements of the user's wrist, hand, or fingers. The features may be, for example, the temporal pattern of local minima/maxima in a signal, the magnitude of a signal between local minima/maxima, or the peak components of the signal's spectrogram.

In block 760, it is determined whether the identified features match a gestural action. For example, the wearable device may store a number of features in correlation with particular gestures, such as squeeze, tap, hold, etc. Accordingly, the gesture made by the user may be determined based on the identified features of the filtered signal. If no match is identified, the method 700 may return to block 710.

If the features match a gestural action, in block 770 an interface operation corresponding to the matched gestural action is performed. The interface operation may be, for example, any operation to control a function of the wearable device. For example, the interface operation may adjust a volume, scroll through a menu or lines of text, change information displayed, change audio emitted, turn on a microphone, pair or unpair with other wireless devices, etc.

In some examples, identifying the gestural action may simply include determining an operation to be performed. For example, the identified features may be matched with an operation, without first identifying the motion that caused such features.

The foregoing systems and methods are beneficial in that they enable one-handed interactions with smartwatches and other wearable electronic devices. Sensors on the smartwatch are configured to detect one-handed gestures in real time, without requirement of per-user or per-session calibration. The gestures may be simple, one-shot, discrete, and serve various use cases. In this regard, user experience is improved because users will not have to stop an activity or free an opposite hand to enter input to the smartwatch.

Unless otherwise stated, the foregoing alternative examples are not mutually exclusive, but may be implemented in various combinations to achieve unique advantages. As these and other variations and combinations of the features discussed above can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including” and the like, should not be interpreted as limiting the subject matter of the claims to the specific examples; rather, the examples are intended to illustrate only one of many possible embodiments. Further, the same reference numbers in different drawings can identify the same or similar elements.

1. A method for detecting hand motions by a wearable device, comprising:
emitting, by a light source, light towards a user's skin;
receiving, by an optical sensor, reflected light;
reading, by one or more processors, raw sensor data from the optical sensor;
filtering, by the one or more processors, the raw sensor data to reduce noise;
identifying, by the one or more processors, features in the filtered signal that correspond to movements of the wrist, hand, or fingers;
matching, by the one or more processors, the identified features to a specific gestural action; and
performing, by the one or more processors, an interface operation corresponding to the specific gestural action.

2. The method of claim 1, wherein the wearable device is a smartwatch worn on the user's arm.

3. The method of claim 2, wherein the light is reflected off target and non-target objects in the user's arm.

4. The method of claim 3, wherein the target objects include hemoglobin.

5. The method of claim 1, wherein the filtering comprises high-pass filtering and low-pass filtering.

6. The method of claim 5, wherein the filtering further comprises a median filter adapted to reduce spikes in the raw sensor data.

7. The method of claim 1, further comprising:
receiving sensor data from an inertial measurement unit (IMU);
wherein the filtering comprises using the sensor data from the IMU to filter out noise from the raw sensor data from the optical sensor.

8. The method of claim 1, wherein identifying features comprises identifying changing points in the filtered sensor data.

9. The method of claim 1, wherein the matching comprises matching the identified features to recorded features for particular gestures.

10. The method of claim 1, wherein the interface operation controls a function of the wearable electronic device.

11. A wearable electronic device adapted to receive input from hand gesture of an arm on which the device is worn, comprising:
a light source configured to light towards the user's arm;
an optical sensor adapted to receive reflected light from the user's arm;
a memory storing information regarding gestural actions and interface operations;
one or more processors in communication with the optical sensor and the memory, the one or more processors configured to:
receive raw sensor data from the optical sensor;
filter the raw sensor data to reduce noise;
identify features in the filtered signal that correspond to movements of the wrist, hand, or fingers of the arm wearing the device;
match the identified features to a specific gestural action; and
perform an interface operation corresponding to the specific gestural action.

12. The wearable electronic device of claim 11, wherein the wearable electronic device is a smartwatch, and the light is reflected off target and non-target objects in the user's arm.

13. The wearable electronic device of claim 12, wherein the target objects include hemoglobin.

14. The wearable electronic device of claim 11, wherein the filtering comprises high-pass filtering and low-pass filtering.

15. The wearable electronic device of claim 14, wherein the filtering further comprises a median filter adapted to reduce spikes in the raw sensor data.

16. The wearable electronic device of claim 11, further comprising:
an inertial measurement unit (IMU) adapted to detect motions of the user's arm when the wearable electronic device is worn;
wherein the filtering comprises using readings from the IMU to filter out noise from the raw sensor data from the optical sensor.

17. The wearable electronic device of claim 11, wherein identifying features comprises identifying changing points in the filtered sensor data.

18. The wearable electronic device of claim 11, wherein the matching comprises matching the identified features to recorded features for particular gestures.

19. The wearable electronic device of claim 1, wherein the interface operation controls a function of the wearable electronic device.

20. A system, comprising:
a photoplethysmogram (PPG) sensor adapted to be positioned in close proximity to a user's arm;
an inertial measurement unit (IMU) adapted to be positioned in close proximity to the user's arm;
wherein each of the PPG sensor and the IMU are configured to receive data signals measuring movements of the user's arm; and
one or more processing units, configured to process the received data signals, such processing including extracting features indicating particular hand gestures, and correlate the detected features with input operations for the one or more processors.
